(19) United States
(12) Patent Application Publication
(10) Pub. No.: US 2022/0023509 A1
(43) Pub. Date: Jan. 27, 2022

(54) METHOD OF USING AN INDWELLING CATHETER APPARATUS INCLUDING DISPOSABLE ANTI-MICROBIAL SLEEVE

(71) Applicant: Aaron R. Garside, Harrogate, TN (US)

(72) Inventor: Aaron R. Garside, Harrogate, TN (US)

(21) Appl. No.: 17/363,475

(22) Filed: Jun. 30, 2021

Related U.S. Application Data
(63) Continuation-in-part of application No. 16/937,649, filed on Jul. 24, 2020, now abandoned.

(51) Int. Cl. A61L 29/16; A61M 25/00; A61L 29/04

(52) U.S. Cl. CPC A61L 29/16; A61M 25/0017; A61L 2300/404; A61M 2205/0205; A61L 29/042

(57) ABSTRACT

A method of using a disposable catheter sleeve to prevent infection. A method of using an indwelling catheter apparatus including a catheter tube comprising a first end for insertion into a patient's body and a disposable catheter sleeve wrapped around the catheter tube proximate to the first end of the catheter tube to prevent infection.

### CROSS-REFERENCE(S) TO RELATED APPLICATION(S)

This application claims priority to and is a continuation-in-part of pending U.S. nonprovisional patent application Ser. No. 16/937,649 entitled “INDWELLING CATHETER APPARATUS INCLUDING DISPOSABLE ANTI-MICROBIAL SLEEVE” which was filed on Jul. 24, 2020, the entirety of which is incorporated herein by reference.

### FIELD

This disclosure relates to the field of indwelling catheters. More particularly, this disclosure relates to an indwelling catheter apparatus including a removable, disposable antimicrobial sleeve.

### BACKGROUND

One type of catheter technology in the medical fields includes indwelling catheters, some of which are often referred to in the medical field as “Foley” catheters. A distal end of such catheters is inserted into a patient's urethra into the patient's bladder. Often, patients that have catheters inserted also suffer from incontinence. When a bowel movement occurs, fecal matter often winds up on a catheter tube proximate to where such catheter tube is inserted into the patient's urethra. As such, catheter tubes near a patient's urethra must be cleaned often to remove such fecal matter. When traces of fecal matter remain on the catheter tube, bacteria grow or move up along the catheter tube into a patient's urethra, thereby causing a urinary tract and/or bladder infection. In extreme cases, such infections can lead to death of the patient. In less extreme cases, hospitals are tasked with paying for any such infection caused by the conditions of a patient's catheter tube when such tubes are not promptly and properly cleaned to avoid such infections.

What is needed, therefore, is a device and method to help keep catheter tubes clean and as sterile as possible and also making the cleaning of catheter tubes easier.

### SUMMARY

The above and other needs are met by an indwelling catheter apparatus comprising a catheter tube comprising a first end for insertion into a patient's body; and a disposable catheter sleeve wrapped around the catheter tube proximate to the first end of the catheter tube. The disposable catheter sleeve may further include a sleeve body including a first layer of elastic material. The first layer may include an antimicrobial agent. The sleeve body may also include a second layer (an internal layer) including an antimicrobial agent. The sleeve body may also include a third layer (an outer layer) including an antimicrobial agent. The disposable catheter sleeve may further include a lengthwise slit along the disposable catheter sleeve for wrapping the disposable catheter sleeve around the catheter tube. The disposable catheter sleeve may further include an aperture along the disposable catheter sleeve for a fluid port.

In another aspect, embodiments of the disclosure provide a disposable catheter sleeve for wrapping around a catheter tube proximate to where a catheter tube is inserted into a patient, the disposable catheter sleeve comprising an elastic sleeve body including a first end and a second end; and a lengthwise slit along the sleeve body from the first end to the second end. The sleeve body may further include a first layer of elastic material. The first layer may include an antimicrobial agent. The sleeve body may also include a second layer (an internal layer) of antimicrobial material extending at least partially along a length of the first layer. The sleeve body may also include a third layer (an outer layer) of antimicrobial material extending at least partially along a length of the first layer. The disposable catheter sleeve may further include an aperture along the elastic sleeve body. The first layer is preferably made of a material such as a polymer-based material such as plastic. Specific examples of materials that can be used include soft plastic, silicone rubber and latex. Other examples include nitinol, nylon, polyurethane, thermoplastic elastomers and polyethylene terephthalate. The antimicrobial agent(s) used in the second layer and/or the third layer may include for example, a quaternary ammonium compound, chlorhexidine, chloroxylenol, a fungal inhibitor, and/or an antimicrobial peptide (AMP). The antimicrobial agent may include a bactericide such as, for example, gentamicin, cephalothin, carbenicillin, amoxicillin, cefamandol, tobramycin, and vancomycin.

In another aspect, embodiments of the disclosure provide a method of using an indwelling catheter apparatus including a disposable catheter sleeve to prevent to prevent infection, the method comprising inserting a first end of a catheter tube into a medical patient's urethra; and wrapping a first disposable catheter sleeve around the catheter tube at a location proximate to an insertion point of the catheter tube into the medical patient's body wherein the first disposable catheter sleeve comprises a first disposable catheter sleeve body including a first layer and wherein the first disposable catheter sleeve body includes a first disposable catheter sleeve body first end and a first disposable catheter sleeve body second end. The first disposable catheter sleeve may further comprise an antimicrobial agent. The first disposable catheter sleeve may further comprise a second layer including an antimicrobial agent wherein the second layer is disposed internal to the first layer. The first disposable catheter sleeve may further comprise a third layer including an antimicrobial agent wherein the third layer is disposed external to the first layer. The first disposable catheter sleeve may further comprise a first lengthwise slit along the first disposable catheter sleeve from the first disposable catheter sleeve first end to the first disposable catheter sleeve second end for wrapping the first disposable catheter sleeve around the catheter tube. The first disposable catheter sleeve may further comprise an aperture along the disposable catheter sleeve for circumnavigating a fluid port. The first layer may be made of a material comprising an elastic polymer-based material. The antimicrobial agent may comprise a member selected from the group consisting of a quaternary ammonium compound, chlorhexidine, chloroxylenol, a fungal inhibitor, and an antimicrobial peptide. The antimicrobial agent may comprise a bactericide selected from the group consisting of gentamicin, cephalothin, carbenicillin, amoxicillin, cefamandol, tobramycin, and vancomycin. The method may further comprise removing the first disposable catheter sleeve from the catheter tube; and wrapping a second disposable catheter sleeve around the catheter tube at the location proximate to an insertion point of the catheter tube into the medical patient's body wherein the second disposable catheter sleeve comprises a second disposable catheter sleeve body including a first layer and wherein the second disposable catheter sleeve body includes a second disposable catheter sleeve body first end and a second disposable catheter sleeve body second end.

The summary provided herein is intended to provide examples of particular disclosed embodiments and is not intended to cover all potential embodiments or combinations of embodiments. Therefore, this summary is not intended to limit the scope of the invention disclosure in any way, a function which is reserved for the appended claims.

### BRIEF DESCRIPTION OF THE DRAWINGS

Further features, aspects, and advantages of the present disclosure will become better understood by reference to the following detailed description, appended claims, and accompanying figures, wherein elements are not to scale so as to more clearly show the details, wherein like reference numbers indicate like elements throughout the several views, and wherein:

FIG. 1 shows a perspective view of a disposable protective sleeve for use along a catheter tube where a catheter is inserted into a patient;

FIG. 2 shows a plan view of the disposable protective sleeve shown in FIG. 1;

FIG. 3 shows an end view of the disposable protective sleeve shown in FIG. 1 and FIG. 2;

FIG. 4 shows a perspective view of a disposable protective sleeve including an inner antimicrobial layer for use along a catheter tube where a catheter is inserted into a patient;

FIG. 5 shows a plan view of the disposable protective sleeve shown in FIG. 4;

FIG. 6 shows an end view of the disposable protective sleeve shown in FIG. 4 and FIG. 5;

FIG. 7 shows a perspective view of a disposable protective sleeve including an inner antimicrobial layer and an outer antimicrobial layer for use along a catheter tube where a catheter is inserted into a patient;

FIG. 8 shows a plan view of the disposable protective sleeve shown in FIG. 7;

FIG. 9 shows an end view of the disposable protective sleeve shown in FIG. 7 and FIG. 8;

FIG. 10 shows an image of an indwelling catheter apparatus including a protective sleeve from FIGS. 4-6 engaged therewith at a location proximate to where the catheter apparatus would be inserted into a patient.

FIG. 11 shows a perspective view of a disposable protective sleeve including an inner antimicrobial layer for use along a catheter tube where a catheter is inserted into a patient and wherein the sleeve includes an aperture;

FIG. 12 shows a plan view of the disposable protective sleeve shown in FIG. 11;

FIG. 13 shows an end view of the disposable protective sleeve shown in FIG. 11 and FIG. 12;

FIG. 14 shows an image of an indwelling catheter apparatus including a protective sleeve shown in FIGS. 11-13 engaged therewith at a location proximate to where the catheter apparatus would be inserted into a patient; and

FIG. 15 shows a flowchart describing a method of using the indwelling catheter apparatuses and disposable indwelling catheter sleeves described herein.

The figures are provided to illustrate concepts of the invention disclosure and are not intended to embody all potential embodiments of the invention. Therefore, the figures are not intended to limit the scope of the invention disclosure in any way, a function which is reserved for the appended claims.

### DETAILED DESCRIPTION

Various terms used herein are intended to have particular meanings. Some of these terms are defined below for the purpose of clarity. The definitions given below are meant to cover all forms of the words being defined (e.g., singular, plural, present tense, past tense). If the definition of any term below diverges from the commonly understood and/or dictionary definition of such term, the definitions below control.

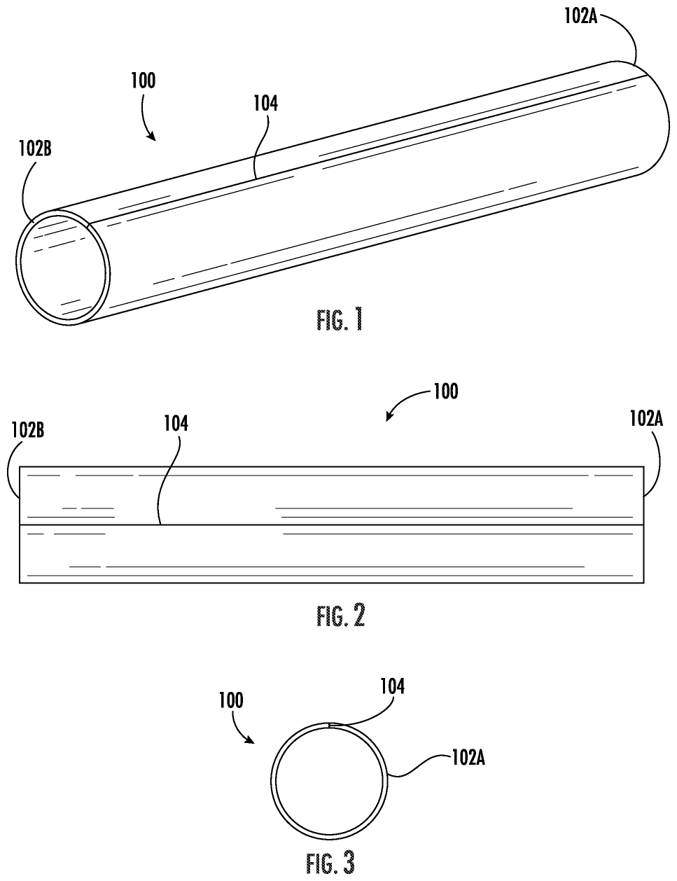

FIGS. 1-3 shows an embodiment of a disposable catheter sleeve 100 including a first end 102A and a second end 102B. The sleeve 100 includes a slit 104 cut lengthwise along the sleeve 100 from the first end 102A to the second end 102B. FIG. 2 shows a plan view of the sleeve 100 showing the slit 104. The sleeve may include a single layer 106 of elastic material such as for example, a polymer-based material such as plastic. Specific examples of materials that can be used include soft plastic, silicone rubber and latex. Other examples include nitinol, nylon, polyurethane, thermoplastic elastomers and polyethylene terephthalate. FIG. 3 shows an end view of the sleeve. Preferably, the material selected for the sleeve preferably includes an antimicrobial agent. The antimicrobial agent may include, for example, a quaternary ammonium compound, chlorhexidine, chloroxylenol, a fungal inhibitor, and/or an antimicrobial peptide (AMP). The antimicrobial agent may include a bactericide such as, for example, gentamicin, cephalothin, carbenicillin, amoxicillin, cefamandol, tobramycin, and vancomycin.

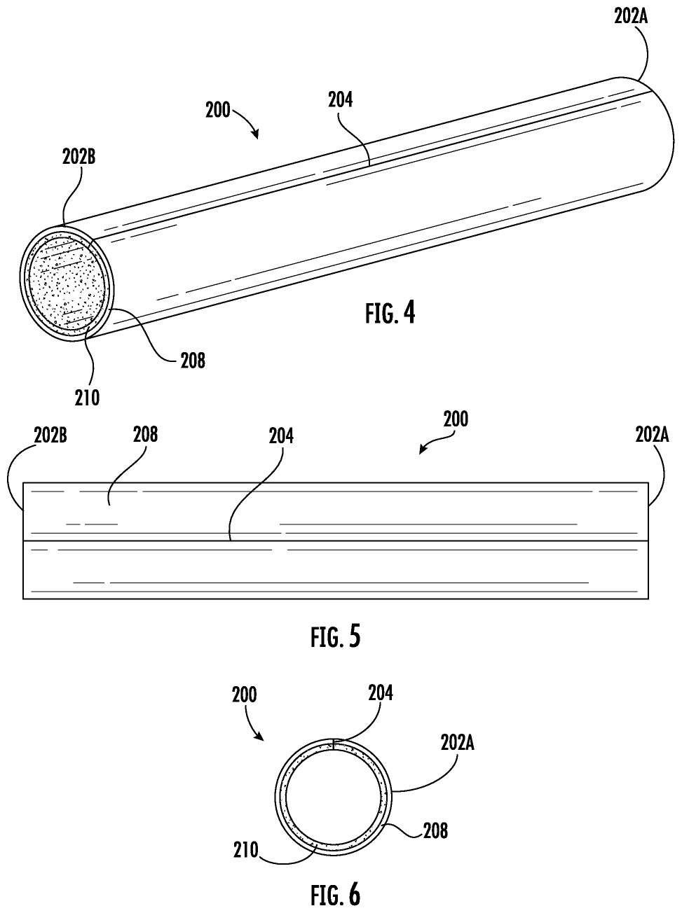

In an alternative embodiment shown in FIGS. 4-6, a disposable catheter sleeve 200 is shown including a first end 202A and a second end 202B. The sleeve 200 includes a slit 204 cut lengthwise along the sleeve 200 from the first end 202A to the second end 202B. FIG. 5 shows a plan view of the sleeve 200 showing the slit 204. The sleeve 200 preferably includes a first layer 208 made of an elastic material such as a polymer-based material such as plastic and a second layer 210—an antimicrobial inner layer including an antimicrobial agent. Specific examples of materials that can be used for the first layer 208 include soft plastic, silicone rubber and latex. Other examples include nitinol, nylon, polyurethane, thermoplastic elastomers and polyethylene terephthalate. The antimicrobial agent may include, for example, a quaternary ammonium compound, chlorhexidine, chloroxylenol, a fungal inhibitor, and/or an antimicrobial peptide (AMP). The antimicrobial agent may include a bactericide such as, for example, gentamicin, cephalothin, carbenicillin, amoxicillin, cefamandol, tobramycin, and vancomycin. FIG. 6 shows an end view of the sleeve 200 showing the first layer 208 and the second layer 210. In some embodiments the antimicrobial inner layer 210 extends along the entire length of the first layer 208 and in other embodiments the antimicrobial inner layer 210 only extends part of the length of the outer layer 208.

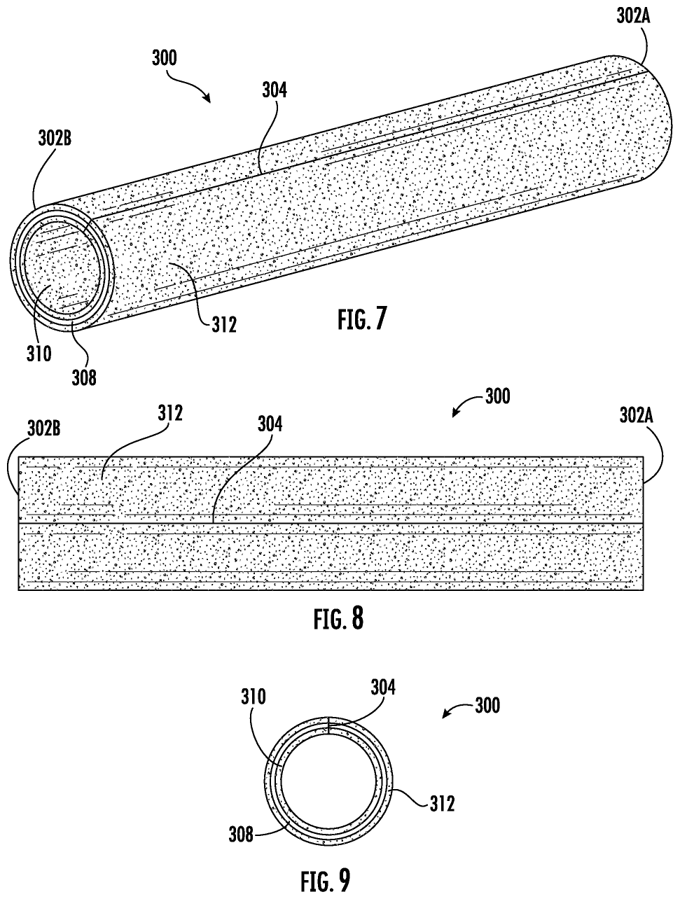

In yet another embodiment shown in FIGS. 7-9, a disposable catheter sleeve 300 is shown including a first end 302A and a second end 302B. The sleeve 300 includes a slit 304 cut lengthwise along the sleeve 300 from the first end 302A to the second end 302B. FIG. 8 shows a plan view of the sleeve 300 showing the slit 304. The sleeve 300 preferably includes a first layer 308 made of an elastic material such as a polymer-based material such as plastic. Specific examples of materials that can be used for the first layer 308 include soft plastic, silicone rubber and latex. Other examples include nitinol, nylon, polyurethane, thermoplastic elastomers and polyethylene terephthalate. The sleeve 300 further includes a second layer 310—an antimicrobial inner layer including an antimicrobial agent. The antimicrobial agent may include, for example, a quaternary ammonium compound, chlorhexidine, chloroxylenol, a fungal inhibitor, and/or an antimicrobial peptide (AMP). The antimicrobial agent may include a bactericide such as, for example, gentamicin, cephalothin, carbenicillin, amoxicillin, cefamandol, tobramycin, and vancomycin. The sleeve 300 further includes a third layer 312—an antimicrobial outer layer including an antimicrobial agent such as the antimicrobial agent(s) used in the second layer 310. FIG. 9 shows an end view of the sleeve 300 showing the first layer 308, the second layer 310, and the third layer 312. In some embodiments the antimicrobial inner layer 310 and the antimicrobial outer layer 312 extend along the entire length of the first layer 308 and in other embodiments the antimicrobial inner layer 310 and the antimicrobial outer layer 312 only extend part of the length of the first layer 308.

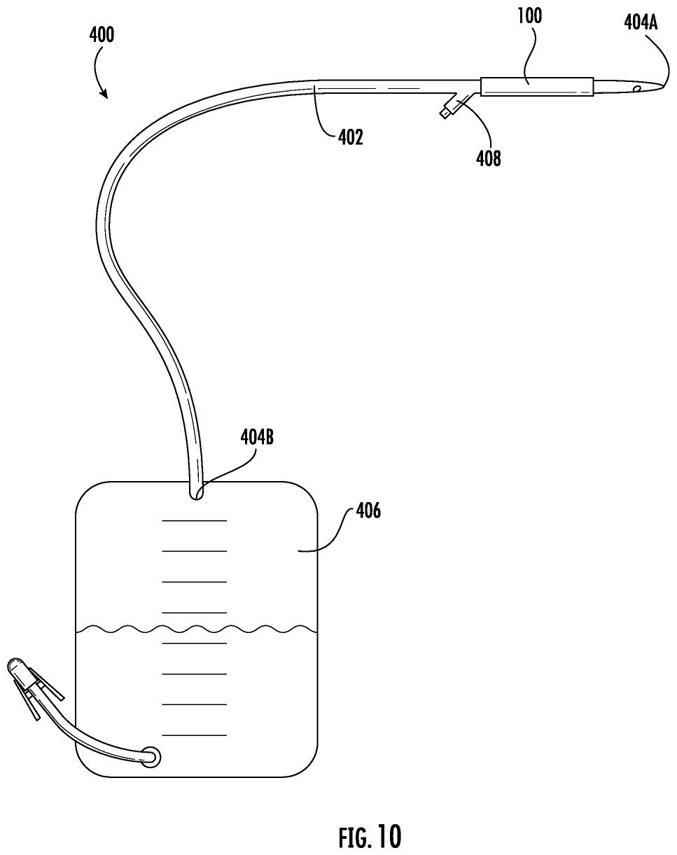

The disposable catheter sleeves described herein (100, 200, or 300) are preferably used on an indwelling catheter apparatus 400 shown in FIG. 10. The indwelling catheter apparatus 400 includes a catheter tube 402 including a first end 404A and a second end 404B. The first end 404A is configured for insertion into a patient's body. In the case of a Foley catheter, such insertion occurs in the patient's urethra. The disposable catheter sleeve (100, 200, or 300) is preferably wrapped around the catheter tube 402 at a location proximate to the interface between a patient's body and the catheter tube 402. As such, the disposable catheter sleeve (100, 200 or 300) is typically not situated at the first end 404A of the catheter tube 402 but is some distance from first end 404A of the catheter tube 402. Because patients may vary in size, the disposable catheter sleeve (100, 200 or 300) is preferably added to the catheter tube 402 after the first end 404A of the catheter tube 402 has been inserted into a patient's urethra. The indwelling catheter apparatus 400 may further include the second end 404B of the catheter tube 402 in communication with a liquid collection bag 406, The indwelling catheter apparatus 400 typically includes a port 408 for access to the catheter tube 402. Such port can be used, for example, as a balloon port for inflating a balloon near the first end 404A of the catheter tube 402.

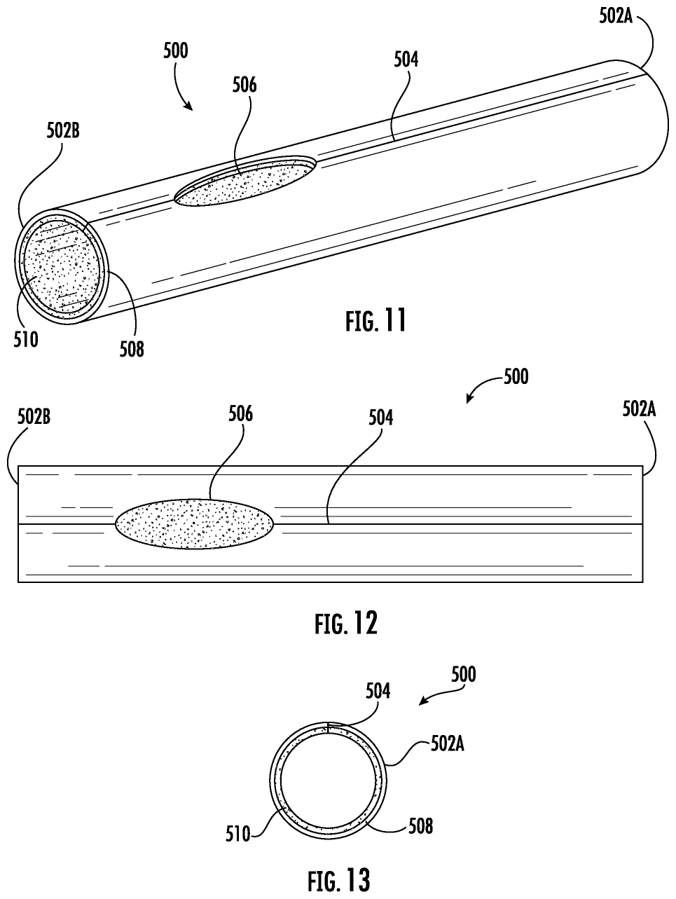

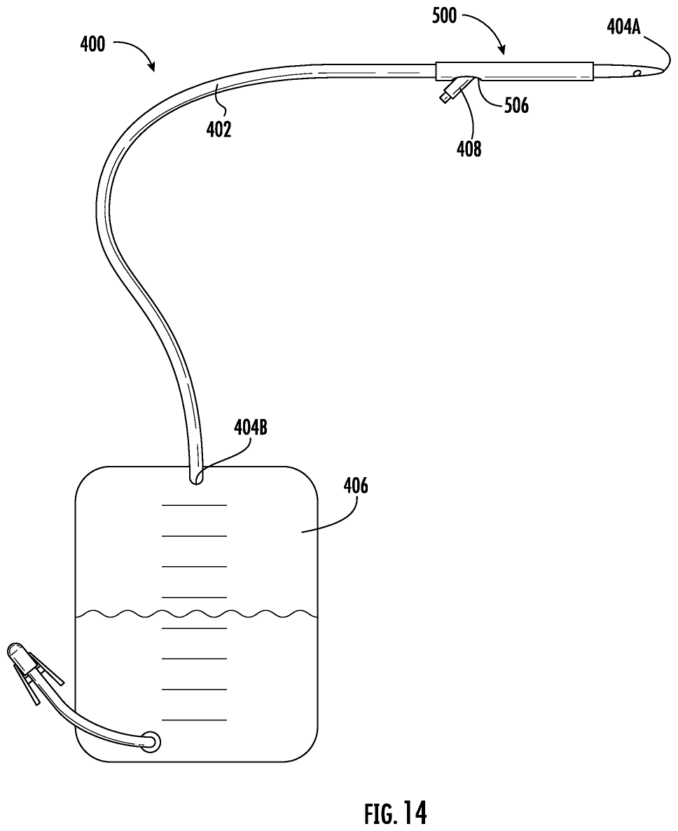

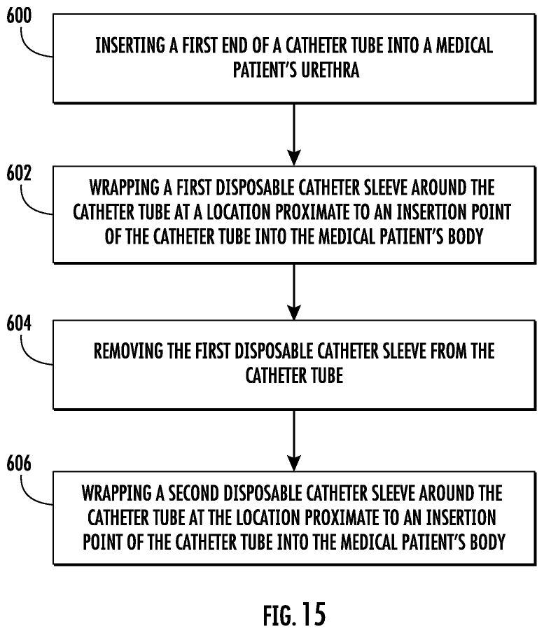

In an alternative embodiment shown in FIGS. 11-13, a disposable catheter sleeve 500 is shown including a first end 502A and a second end 502B. The sleeve 500 includes a slit 504 cut lengthwise along the sleeve 500 from the first end 502A to the second end 502B and an aperture 506 along the sleeve for a balloon port or other additional port. In this embodiment, and others like it, the sleeve 500 is longer, thereby requiring the aperture 506 for the sleeve 500 to fit around a catheter tube and circumnavigate any port such as the port 408 shown below in FIG. 14. FIG. 12 shows a plan view of the sleeve 500 showing the slit 504. The sleeve 500 preferably includes a first layer 508 made of an elastic material such as a polymer-based material such as plastic and a second layer 510—an antimicrobial inner layer including an antimicrobial agent. In other similar embodiments including an aperture like aperture 506, such sleeves may include only the first layer 508 and others may include the first layer 508, the second layer 510, and a third antimicrobial outer layer similar to the third layer 312 shown in the embodiment in FIGS. 7-9. Specific examples of materials that can be used for the first layer 508 include soft plastic, silicone rubber and latex. Other examples include nitinol, nylon, polyurethane, thermoplastic elastomers and polyethylene terephthalate. The antimicrobial agent may include, for example, a quaternary ammonium compound, chlorhexidine, chloroxylenol, a fungal inhibitor, and/or an antimicrobial peptide (AMP). The antimicrobial agent may include a bactericide such as, for example, gentamicin, cephalothin, carbenicillin, amoxicillin, cefamandol, tobramycin, and vancomycin. FIG. 13 shows an end view of the sleeve 500 showing the first layer 508 and the second layer 510. In some embodiments the antimicrobial inner layer 510 extends along the entire length of the first layer 508 and in other embodiments the antimicrobial inner layer 510 only extends part of the length of the outer layer 508.

The disposable catheter sleeve 500 and related embodiments are preferably used on the indwelling catheter apparatus 400 shown in FIG. 14. The indwelling catheter apparatus 400 includes the catheter tube 402 including the first end 404A and the second end 404B. The first end 404A is configured for insertion into a patient's body. The disposable catheter sleeve 500 is preferably wrapped around the catheter tube 402 at a location proximate to the interface between a patient's body and the catheter tube 402. As such, the disposable catheter sleeve 500 is typically not situated at the first end 404A of the catheter tube 402 but is some distance from first end 404A of the catheter tube 402. Because patients may vary in size, the disposable catheter sleeve 500 is preferably added to the catheter tube 402 after the first end 404A of the catheter tube 402 has been inserted into a patient's urethra. The indwelling catheter apparatus 400 may further include the second end 404B of the catheter tube 402 in communication with a liquid collection bag 406. The catheter sleeve 500 includes the aperture 506 so that the sleeve 500 can circumnavigate the port 408 extending out of the catheter tube 402.

In addition to Foley catheters, the disposable catheter sleeve may also be used with other indwelling catheters such as, for example, suprapubic catheters.

In addition to the embodiments of catheter sleeves described above, also disclosed herein is a method of using such catheter sleeves. The method preferably includes inserting a first end of a catheter tube into a medical patient's urethra (600), wrapping a first disposable catheter sleeve around the catheter tube at a location proximate to an insertion point of the catheter tube into the medical patient's body (602). The first disposable catheter sleeve may be any of the catheter sleeves described herein or variations thereof including any features of such sleeves described above. The method may further include removing the first disposable catheter sleeve from the catheter tube (604) and wrapping a second disposable catheter sleeve around the catheter tube at the location proximate to an insertion point of the catheter tube into the medical patient's body (606). The second disposable catheter sleeve may be any of the catheter sleeves described herein or variations thereof including any features of such sleeves described above.

The previously described embodiments of the present disclosure have many advantages including use of the disposable catheter sleeve which helps keep the catheter tube 402 clean. If a patient has a bowel movement and fecal matter ends up on the catheter sleeve, the disposable catheter sleeve and associated fecal matter can be removed with the fecal matter and a new disposable catheter sleeve can be added quickly in place of the old one. To the extent that any fecal matter or other material including bacteria or which increases the risk of bacterial growth gets beneath any disposable catheter sleeve including an antimicrobial inner layer, the antimicrobial inner layer helps to kill bacteria and minimize bacterial growth before the catheter tube 402 can be cleaned and a new disposable catheter sleeve applied to the catheter tube 402. By killing and minimizing bacteria, these various features decrease the chance for infection of the patient, thereby decreasing the risk for serious complications from infection. The various features also help lower hospital operating costs for handling infections because the infection rate will be decreased by use of the disposable catheter sleeve and other antimicrobial features.

The foregoing description of preferred embodiments of the present disclosure has been presented for purposes of illustration and description. The described preferred embodiments are not intended to be exhaustive or to limit the scope of the disclosure to the precise form(s) disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the concepts revealed in the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

Any element in a claim that does not explicitly state “means for” performing a specified function, or “step for” performing a specific function, is not to be interpreted as a “means” or “step” clause as specified in 35 U.S.C. § 112, 116. In particular, the use of “step of” in the claims herein is not intended to invoke the provisions of 35 U.S.C. § 112, 116.

What is claimed is:

1. A method of using an indwelling catheter apparatus including a disposable catheter sleeve to prevent to prevent infection, the method comprising:
a. inserting a first end of a catheter tube into a medical patient's urethra;
b. wrapping a first disposable catheter sleeve around the catheter tube at a location proximate to an insertion point of the catheter tube into the medical patient's body wherein the first disposable catheter sleeve comprises a first disposable catheter sleeve body including a first layer and wherein the first disposable catheter sleeve body includes a first disposable catheter sleeve body first end and a first disposable catheter sleeve body second end.

2. The method of claim 1 wherein the first disposable catheter sleeve further comprises an antimicrobial agent.

3. The method of claim 1 wherein the first disposable catheter sleeve further comprises a second layer including an antimicrobial agent wherein the second layer is disposed internal to the first layer.

4. The method of claim 3 wherein the first disposable catheter sleeve further comprises a third layer including an antimicrobial agent wherein the third layer is disposed external to the first layer.

5. The method of claim 1 wherein the first disposable catheter sleeve further comprises a first lengthwise slit along the first disposable catheter sleeve from the first disposable catheter sleeve first end to the first disposable catheter sleeve second end for wrapping the first disposable catheter sleeve around the catheter tube.

6. The method of claim 3 wherein the first disposable catheter sleeve further comprises a first lengthwise slit along the first disposable catheter sleeve from the first disposable catheter sleeve first end to the first disposable catheter sleeve second end for wrapping the first disposable catheter sleeve around the catheter tube.

7. The method of claim 4 wherein the first disposable catheter sleeve further comprises a first lengthwise slit along the first disposable catheter sleeve from the first disposable catheter sleeve first end to the first disposable catheter sleeve second end for wrapping the first disposable catheter sleeve around the catheter tube.

8. The method of claim 1 wherein the first disposable catheter sleeve further comprises an aperture along the disposable catheter sleeve for circumnavigating a fluid port.

9. The method of claim 1 wherein the first layer is made of a material comprising an elastic polymer-based material.

10. The method of claim 2 wherein the antimicrobial agent comprises a member selected from the group consisting of a quaternary ammonium compound, chlorhexidine, chloroxylenol, a fungal inhibitor, and an antimicrobial peptide.

11. The method of claim 2 wherein the antimicrobial agent comprises a bactericide selected from the group consisting of gentamicin, cephalothin, carbenicillin, amoxicillin, cefamandol, tobramycin, and vancomycin.

12. The method of claim 1 further comprising:
c. removing the first disposable catheter sleeve from the catheter tube; and
d. wrapping a second disposable catheter sleeve around the catheter tube at the location proximate to an insertion point of the catheter tube into the medical patient's body wherein the second disposable catheter sleeve comprises a second disposable catheter sleeve body including a first layer and wherein the second disposable catheter sleeve body includes a second disposable catheter sleeve body first end and a second disposable catheter sleeve body second end.
